(19) United States
(12) Patent Application Publication
(10) Pub. No.: US 2025/0002843 A1
(43) Pub. Date: Jan. 2, 2025

(54) DERIVATION OF NAÏVE BOVINE EMBRYONIC STEM CELLS

(71) Applicant: The Semex Alliance, Guelph (CA)

(72) Inventors: Remi Labrecque, St-Pie (CA); Si-Jung Jang, Granby (CA)

(21) Appl. No.: 18/709,061

(22) PCT Filed: Nov. 11, 2022
(86) PCT No.: PCT/CA2022/051664

§ 371 Date: May 10, 2024

Related U.S. Application Data
(60) Provisional application No. 63/278,751, filed on Nov. 12, 2021.

(51) Int. Cl. C12N 5/0735; C12N 5/073

(52) U.S. Cl. CPC C12N 5/0606; C12N 5/0604; C12N 2501/01; C12N 2501/16; C12N 2501/235; C12N 2501/33; C12N 2501/415; C12N 2501/727; C12N 2533/30; C12N 2533/54; C12N 2533/90

(57) ABSTRACT

The disclosure relates to methods, products and compositions useful for the derivation of naïve bovine embryonic stem cells. ECM-coated substrates comprising a positively charged biocompatible polymer are described that allow for embryo attachment and outgrowth formation. Also provided is outgrowth medium suitable for culturing bovine embryos and deriving naive embryonic stem cells. The naïve bovine embryonic stem cells may be used for in vitro breeding programs including the multiplication of preimplantation embryos with desirable genetic characteristics, deriving primordial germ cells/gametes for in vitro breeding programs, and developing and delivering veterinary medical biologicals and therapeutics.

### CROSS-REFERENCE TO RELATED APPLICATIONS

This application claims the benefit of priority to U.S. Provisional Application No. 63/278,751, filed Nov. 12, 2021, the contents of which are incorporated herein by reference in their entirety.

### FIELD

The present disclosure relates to bovine stem cells and more specifically to naïve bovine embryonic stem cells, associated methods, and compositions.

### INTRODUCTION

Naïve embryonic stem cells can differentiate into all types of cells in the body, including extraembryonic cells such as trophoblast stem cells and extraembryonic endodermal lineage cells. Mouse blastocyst-like structures (also called iblastoids) have been generated by differentiating Expanded Pluripotent Stem (EPS) Cells (Li et al., 2019) and more recently human iblastoid structures have been generated using either naïve pluripotent stem cells derived from embryonic stem cells (Yu et al., 2021) or induced Pluripotent Stem cells (iPS, Liu et al., 2021). However, in contrast to the mouse or human, the derivation of bovine naïve stem cells has not been reported yet.

There are very few reports of bovine primed embryonic stem cells derived from pre-implantation embryos and more specifically the Inner Cell Mass (ICM) structure of the embryo (Bogliotti et al. 2018; Soto et al., 2021). However, primed ESCs do not differentiate into extraembryonic cells, thus cannot be used directly for the generation of iblastoid structures. Furthermore, primed ESCs cannot be used efficiently for germ cell differentiation for use in in vitro breeding (Hou et al., 2018). Naïve embryonic stem cells which represent the ground state of pluripotency (pre-implantation ICM), can differentiate into germ cells more efficiently than primed ES (De Los Angeles, 2019).

In contrast to using a nuclear transfer (cloning) approach which is both labor intensive and generally limited by a low embryo production rate (˜20-30% of blastocyst formation), the derivation of bovine naïve stem cells may facilitate the efficient multiplication of embryos with desirable characteristics. Desirable genetic characteristics in embryos may arise as a result of processes such as meiosis, mutation, and the immigration of genes, which can occur naturally, may be generated using assisted reproductive technologies such as Smith et al., WO 2020/168422, or by genetic modification. In addition, animal and veterinary sciences can realize a broad array of benefits from naïve stem cell technology by providing an efficient platform for producing genetically modified animals at scale, delivering important constituent technologies for in vitro breeding programs, and enabling the development and delivery of advanced veterinary medical biologics and therapeutics. There remains a need for naïve bovine embryonic stem cells and associated methods for iblastoid production.

### SUMMARY

Described herein are materials and methods useful for achieving attachment and outgrowth formation using bovine embryos, such as morula and blastocyst stage embryos, for establishing bovine naïve embryonic stem cells. As demonstrated in the Examples, Zona Pellucida (ZP)-free bovine embryos plated on layered ECM-coated substrates provide greater attachment rates and outgrowth formation compared to embryos plated on conventional ECM-coated substrates.

Furthermore, outgrowth media compositions and associated methods are described which support embryo attachment and outgrowth formation, as well as the propagation of the inner cell mass (ICM) cells from such outgrowths, allowing for the derivation of naïve embryonic stem cells from the ICM. The embodiments described herein are therefore useful for deriving naïve bovine embryonic stem cells and optionally for use in breeding programs such as for generating iblastoid structures, multiplying preimplantation embryos having desirable genetic characteristics, deriving primordial germ cells and/or gametes for in vitro breeding programs, and/or developing and delivering veterinary medical biologicals and therapeutics.

Accordingly, in one aspect there is provided a method for deriving naïve bovine embryonic stem cells. In one embodiment, the method comprises:
providing a Zona Pellucida (ZP)-free bovine embryo comprising naïve bovine embryonic stem cells;
contacting the ZP-free bovine embryo with an extracellular matrix (ECM)-coated substrate, wherein the ECM-coated substrate comprises a substrate comprising a negatively charged substrate surface adjacent to a positively charged biocompatible polymer layer, and a negatively charged ECM layer adjacent to the positively charged biocompatible polymer layer; and
culturing the ZP-free bovine embryo in the presence of outgrowth medium to induce attachment of the ZP-free bovine embryo to the ECM-coated substrate and outgrowth of an inner cell mass (ICM) comprising derived naïve bovine embryonic stem cells.

In an embodiment, the bovine embryo is genetically modified.

In another embodiment, the ZP-free bovine embryo is obtained from a reconstructed diploid embryo.

In one embodiment, the biocompatible polymer is negatively charged at physiological pH. In one embodiment, the biocompatible polymer is type A gelatin.

In an embodiment, the ECM comprises EHS-ECM.

In an embodiment, the substrate comprises polystyrene.

In one embodiment, the outgrowth medium comprises a base medium, and one or more components described herein useful for inducing attachment of the ZP-free embryo to the ECM coated substrate and outgrowth of the ICM in a feeder-free culture system. For example, in one embodiment, the outgrowth medium comprises one or more of: a 1:1 mixture of DMEM/F12 and Neurobasal medium; an N2B27 component; a Wnt activator component; a Wnt inhibitor component; a MEK/ERK inhibitor component; a ROCK inhibitor component; a LIF component; a PKC inhibitor component; and an insulin component. Optionally, the outgrowth medium further comprises an Activin A component.

In an embodiment, the outgrowth medium comprises: the N2B27 component; the Wnt activator component; the Wnt inhibitor component; the MEK/ERK inhibitor component; the ROCK inhibitor component; the LIF component; the Activin A component; the PKC inhibitor; and the insulin component.

In an embodiment, the N2B27 component comprises B27 supplement and N2 supplement, optionally about 1% B27 supplement and about 0.5% N2 supplement; the Wnt activator component comprises CHIR99021, BIO, CHIR-98014, LY2090314, and/or IM-12; the Wnt inhibitor component comprises XAV939, IWR-1, and/or IWP-2; the MEK/ERK inhibitor component comprises PD0325901, Ravoxertinib, GSK1120212, MEK162, PD184352, Trametinib, LY3214996, and/or Ulixertinib; the ROCK inhibitor component comprises Y27632, Thiazovivin, and/or Blebbistatin; the LIF component comprises human LIF; the Activin A comprises human Activin A; the PKC inhibitor comprises Gö6983, Gö6976, LY317615, LY333531, PKC412, GSK690693, Sotrastaurin, Staurosporine, and/or Bisindolylmaleimide; and/or the insulin component comprises insulin.

In an embodiment, the ZP-free bovine embryo is a morula (stage 4); a blastocyst (stage 5); an expanding blastocyst (stage 6); an expanded blastocyst (stage 7); a hatching blastocyst (stage 8) or a hatched blastocyst (stage 9).

In an embodiment, the ZP-free bovine embryo is obtained by enzyme-assisted ZP removal.

In one embodiment, the method comprises obtaining the ZP-free bovine embryo by enzyme-assisted ZP removal.

Also provided herein is a method of enzyme-assisted ZP removal, comprising the steps of: a) providing an embryo; b) contacting the embryo with a protease solution; c) incubating the embryo in the protease solution to partially digest the ZP and obtain a ZP-thinned embryo; d) contacting the ZP-thinned embryo with a protease inactivation medium to inactivate the protease; e) rupturing the ZP; and f) manipulating the embryo to separate the ZP from the embryo.

In an embodiment, the concentration of protease in step c) is about 0.1% to about 0.5%, about 0.2% to 0.3%, or about 0.25%.

In an embodiment, the embryo and protease solution are incubated for between about 30-60 seconds in step c), optionally for about 45 seconds.

In an embodiment, the ZP is ruptured in step e) using a microblade.

In an embodiment, manipulating the embryo in step f) comprises pipetting.

In an embodiment, the method further comprises performing genetic testing to determine one or more genotypes of the ZP-free bovine embryo for one or more biomarkers.

In an embodiment, the method further comprises selecting the ZP-free bovine embryo based on genetic testing for one or more biomarkers.

In an embodiment, the method further comprises performing genetic testing to determine one or more genotypes of the derived naïve bovine embryonic stem cells.

In an embodiment, the ZP-free bovine embryo is obtained from a fresh embryo, optionally a fresh biopsied embryo.

In an embodiment, the embryo of step a) is a genetically modified embryo.

In an embodiment, the ZP-free bovine embryo is obtained from a frozen embryo, optionally a biopsied-frozen embryo.

In an embodiment, the method comprises thawing the frozen embryo and contacting the embryo with a recovery medium.

In an embodiment, the ZP-free bovine embryo is obtained by a method comprising: thawing the frozen embryo; contacting the frozen embryo with the recovery medium; manipulating the embryo to separate the ZP from the embryo in the recovery medium; and incubating the ZP-free bovine embryo in the recovery medium.

In an embodiment, the recovery medium comprises a glycogen synthase kinase 3 (GSK-3) inhibitor, a MEK/ERK kinase inhibitor and a Rho-associated, coiled-coil containing protein kinase (ROCK) inhibitor.

In an embodiment, the recovery medium comprises CHIR99021, PD0325901 and Y27632.

In an embodiment, the method further comprises incubating the ZP-free embryo with an adaptation medium, wherein the adaptation medium comprises a combination of recovery medium and outgrowth medium.

In an embodiment, the adaptation medium comprises a combination of recovery medium and outgrowth medium at a ratio of between about 0.5:1 and 1.5:1 optionally about 1:1.

In another aspect there is provided a naïve bovine stem cell derived using the methods described herein. A further aspect includes use of a naïve bovine stem cell derived using the methods described herein in a breeding scheme or genetic improvement program, and for multiplying preimplantation embryos having desirable genetic characteristics, deriving primordial germ cells and/or gametes for in vitro breeding programs, and/or developing and delivering veterinary medical biologicals and therapeutics.

In another aspect there is provided a method of preparing an extracellular matrix (ECM)-coated substrate, the method comprising: providing a substrate comprising a negatively charged surface; contacting the negatively charged surface with a first solution comprising a biocompatible polymer, wherein the biocompatible polymer is positively charged; incubating the substrate in contact with the first solution such that a layer of the positively charged biocompatible polymer is deposited on the negatively charged surface of the substrate; removing the first solution and optionally washing the substrate; contacting the substrate with a second solution comprising an extracellular matrix (ECM), wherein the ECM is negatively charged; and incubating the substrate in contact with the second solution such that a layer of the negatively charged ECM is deposited on the layer of the positively charged biocompatible polymer.

In an embodiment, the biocompatible polymer comprises type A gelatin.

In an embodiment, the ECM comprises EHS-ECM.

In an embodiment, the substrate comprises polystyrene.

An aspect includes an ECM-coated substrate produced according to the methods described herein.

A further aspect includes an ECM-coated substrate comprising: a substrate comprising a positively charged surface; a layer of a positively charged biocompatible polymer in contact with the negatively charged surface of the substrate; a layer of a negatively charged ECM in contact with the layer of the positively charged biocompatible polymer.

In an embodiment, the biocompatible polymer comprises type A gelatin.

In an embodiment, the ECM comprises EHS-ECM.

In an embodiment, the substrate comprises polystyrene.

An aspect of the disclosure includes use of an ECM-coated substrate described herein for culturing an embryo, optionally to induce ICM outgrowth and/or the derivation of naïve embryonic stem cells.

In an embodiment, the embryo is a bovine embryo, optionally a bovine embryo between day 5 and day 7.

In another embodiment, the bovine embryo is a reconstructed diploid embryo.

In an embodiment, the embryo is a genetically modified embryo.

In another aspect, there is provided a media composition comprising base media and one or more components for culturing an embryo, optionally to induce ICM outgrowth and/or the derivation of naïve embryonic stem cells. In one embodiment, the media composition comprises one or more of: an N2B27 component; a Wnt activator component; a Wnt inhibitor component; a MEK/ERK inhibitor component; a ROCK inhibitor component; a LIF component; a PKC inhibitor; and an insulin component.

Optionally the media composition further comprises an Activin A component.

In an embodiment, the N2B27 component comprises about 1% B27 supplement and about 0.5% N2 supplement; the Wnt activator component comprises CHIR99021, BIO, CHIR-98014, LY2090314, or IM-12; the Wnt inhibitor component comprises XAV939, IWR-1, or IWP-2; the MEK/ERK inhibitor component comprises

PD0325901, Ravoxertinib, GSK1120212, MEK162, PD184352, Trametinib, LY3214996, or Ulixertinib; the ROCK inhibitor component comprises Y27632, Thiazovivin, or Blebbistatin; the LIF component comprises human LIF; the Activin A component comprises human Activin A; the PKC inhibitor comprises Gö6983, Gö6976, LY317615, LY333531, PKC412, GSK690693, Sotrastaurin, Staurosporine, or Bisindolylmaleimide; and/or the insulin component comprises insulin.

A further aspect includes use of a media composition described herein for culturing an embryo to induce ICM outgrowth formation, optionally wherein the embryo is a bovine embryo, a reconstructed diploid bovine embryo, and/or the embryo is a genetically modified embryo.

A further aspect includes a recovery medium comprising: a glycogen synthase kinase 3 (GSK-3) inhibitor, optionally CHIR99021, a MEK/ERK kinase inhibitor, optionally PD0325901, and a Rho-associated, coiled-coil containing protein kinase (ROCK) inhibitor, optionally Y27632.

The preceding section is provided by way of example only and is not intended to be limiting on the scope of the present disclosure and appended claims. Additional objects and advantages associated with the compositions and methods of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages objects and embodiments are expressly included within the scope of the present disclosure. The publications and other materials used herein to illuminate the background of the disclosure, and in particular cases, to provide additional details respecting the practice, are incorporated by reference, and for convenience are listed in the appended reference section.

### DRAWINGS

Further objects, features and advantages of the disclosure will become apparent from the following detailed description taken in conjunction with the accompanying figures showing illustrative embodiments of the disclosure, in which:

FIG. 1 shows results of experiments to determine the attachment and outgrowth rates of embryos plated on different coating materials. (Upper left) Nunc 35 mm culture dish with coating solution, (Upper right) Attachment and outgrowth efficiency depending on coating materials, (Lower left) Nonattached embryo, (Lower right) Attached embryo.

FIG. 2 shows an image of a fabricated dish surface with Geltrex and 30 um pore cell strainer (magnification of image; x100).

FIG. 3 shows a schematic of the chemical structure of a plasma treated Poly styrene dish surface.

FIG. 4 shows an illustration of Layer-by-Layer (LbL) coating.

FIG. 5 shows improved derivation efficiency using a Layer-by-Layer ECM-coated substrate. The LbL ECM-coated substrate exhibited a high and stable attachment rate, TE (trophectodermal cell) and ICM (inner cell mass) growth compared to control protocols using EHS-ECM only.

FIG. 6 shows (Left) a schematic representation of the layout of a protease treatment dish and (Right) an image of protease-treated poor-quality embryos where the ZP was fully removed by the enzymatic treatment.

FIG. 7 shows ZP-free day 6 fresh embryos. All embryos appear healthy after ZP removal.

FIG. 8 shows (Top Left) a schematic representation of the layout of a post-thawing recovery dish, (Top right) and images showing the effect of 2iY media on the recovery of post-thaw embryos. (Bottom) 2iY treated embryos has shown faster recovery than control embryo culture media and higher derivation efficiency as observed by the faster re-expansion of the embryos. 2iY treated embryos has shown bigger size and clear ICM than control group (dashed circle).

FIG. 9 is a graph showing the effect of 2iY on the quality of post-thaw embryos. 2iY treated embryos exhibit more advanced stage of development after post-thawing recovery.

FIG. 10 shows a schematic representation of the layout of the adaptation medium dish #1 and dish #2. Bottom is a graph showing the effect of adaptation time on the derivation of outgrowths.

FIG. 11 is a graph showing derivation results with various types of serum sources. FBS=fetal bovine serum; KOSR=knock-out serum replacement; SR=serum replacement; N2B27=N2 supplement+B27 supplement (see Examples).

FIG. 12 is a schematic showing various Wnt signaling pathway and targets.

FIG. 13 is a graph showing the effect of Forskolin and the dual kinase Wnt pathway modification on the naïve stem cell derivation.

FIG. 14 is a graph showing the effect of IL-6 and SRC inhibitor on the derivation of outgrowths. Bottom is schematic showing various pathways downstream of SRC.

FIG. 15 shows the effect of a MEK inhibitor on the formation of naïve outgrowth.

FIG. 16 is a graph showing the derivation efficiency between naïve stem cell media (left) and an image showing an outgrowth colony derived by t2iLGöY media.

FIG. 17 shows (Left) Endodermal differentiation of outgrowth. ICM colony (arrow) is covered by undifferentiated (arrowhead) and differentiated (star) endodermal cells. (Right) ICM cell colony (chunk of cells which have bright edge) after 1st passaging following insulin addition.

FIG. 18 shows a schematic of the steps involved in deriving cell lines for subsequently generating primordial germ cell lines and gametes and generating multiple genetically identical embryos (also called iblastoids) based on the derivation of naïve bovine embryonic stem cells.

### DESCRIPTION OF VARIOUS EMBODIMENTS

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature described herein may be combined with any other feature or features described herein.

### I. General Definitions

As used herein, the following terms may have meanings ascribed to them below, unless specified otherwise. However, it should be understood that other meanings that are known or understood by those having ordinary skill in the art are also possible, and within the scope of the present disclosure. All publications, patent applications, patents, and other references mentioned herein are incorporated by reference in their entirety. In the case of conflict, the present specification, including definitions, will control. In addition, the materials, methods, and examples are illustrative only and not intended to be limiting.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either both of those included limits are also included in the description.

It must be noted that as used herein and in the appended claims, the singular forms “a”, “an”, and “the” include plural references unless the context clearly dictates otherwise.

All numerical values within the detailed description and the claims herein are modified by “about” or “approximately” the indicated value, and take into account experimental error and variations that would be expected by a person having ordinary skill in the art.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The term “about” as used herein means plus or minus 10%-15%, 5-10%, or optionally about 5% of the number to which reference is being made.

It should be understood that, in certain methods described herein that include more than one step or act, the order of the steps or acts of the method is not necessarily limited to the order in which the steps or acts of the method are recited unless the context indicates otherwise.

It should also be understood that any methods and materials similar or equivalent to those described herein can also be used in the practice or testing of the present disclosure.

### II. Methods

Described herein are methods for naïve embryonic stem cell outgrowth formation and the derivation of naïve embryonic stem cells. As set out in the Examples, the inventors have demonstrated naïve bovine stem cell outgrowth formation in a feeder-free culture system comprising a layered ECM-coated substrate and a specially formulated outgrowth media. The layered ECM-coated substrate and outgrowth media support ex vivo or in vitro attachment and growth of inner cell mass (ICM) cells derived from embryos, such as morula or blastocyst stage (e.g. bovine day 6 or day 7) embryos. The materials and methods described herein are therefore useful for deriving and maintaining naïve bovine embryonic stem cells and optionally for use in breeding programs such as for the multiplication of preimplantation embryos with desirable genetic characteristics and/or the production of iblastoid structures, deriving primordial germ cells and/or gametes for in vitro breeding programs, and developing and delivering veterinary medical biologicals and therapeutics. Desirable genetic characteristics can arise via natural processes or by genetic modification.

Accordingly, in one aspect there is provided a method for deriving naïve bovine embryonic stem cells. In one embodiment, the method comprises:
providing a Zona Pellucida (ZP)-free bovine embryo comprising naïve bovine embryonic stem cells;
contacting the ZP-free bovine embryo with an extracellular matrix (ECM)-coated substrate, wherein the ECM-coated substrate comprises a substrate comprising a negatively charged substrate surface adjacent to a positively charged biocompatible polymer layer, and a negatively charged ECM layer adjacent to the positively charged biocompatible polymer layer; and
culturing the ZP-free bovine embryo in the presence of outgrowth medium to induce attachment of the ZP-free bovine embryo to the ECM-coated substrate and outgrowth of an inner cell mass (ICM) comprising derived naïve bovine embryonic stem cells.

As used herein, the term “naïve embryonic stem cell” is used to refer to embryonic stem cells which substantially retain the molecular characteristics of cells of a morula stage embryo, such as a day-5 or day-6 bovine embryo, where the cells are still in an undifferentiated state. Naïve embryonic stem cells may be found within the inner cell mass (ICM) of a blastocyst (such as a day-7 embryo). Naïve embryonic stem cells may be capable of developing into a complete organism and/or may retain the capacity to give rise to the full complement of adult tissues and/or cell types. Naïve embryonic stem cells are capable of being derived and maintained in an undifferentiated state of self-renewal without the need for exogenously expressed pluripotency factors as opposed to induced Pluripotent Stem Cells (iPSC). Similarly, the term “naïve bovine embryonic stem cell” refers to a naïve embryonic stem cell of bovine origin.

Naïve embryonic stem cells, such as naïve bovine embryonic stem cells, may be derived from sufficiently undifferentiated tissues, such as for example the embryonic cells of an embryo, for example a morula (stage 4); a blastocyst (stage 5); an expanding blastocyst (stage 6); an expanded blastocyst (stage 7); a hatching blastocyst (stage 8) or a hatched blastocyst (stage 9). In one embodiment, naïve bovine embryonic stem cells may be derived from a 3- to 8-day bovine embryo, optionally a 3-, 4-, 5-, 6-, 7-, or 8-day bovine embryo, or a 5- to 7-day bovine embryo. In an embodiment, the bovine embryo is a morula (stage 4); a blastocyst (stage 5); an expanding blastocyst (stage 6); an expanded blastocyst (stage 7); a hatching blastocyst (stage 8) or a hatched blastocyst (stage 9). In an embodiment, the bovine embryo is a 3- to 7-day embryo, optionally a 5-to 7-day embryo or 6- or 7-day embryo. In one embodiment, the embryo is a preimplantation embryo. In one embodiment, the embryo is an embryo that has previously been frozen and/or biopsied. In one embodiment, the embryo is genetically modified. In one embodiment, the embryo has been selected based on genetic testing for one or more biomarkers.

A “genetically modified embryo” refers to an embryo where genomic DNA of the cells in the embryo have been manipulated to express one or more exogenous genes and/or to introduce mutation(s) within endogenous genes or intergenic regions which affects expression or functional activity of one or more endogenous genes or gene products. Examples of successful genetic modifications in bovine embryos have included the introduction of transgenes by microinjection (U.S. Pat. No. 7,067,713) and lentiviral infection (Park, 2007) and most recently genome editing (Bishop and Van Eenennaam, 2020) using transfection and genome editors, such as Zinc Finger Nucleases, transcription activator like effector nuclease (TALEN), and clustered regularly interspaced short palindromic repeat/CRISPR associated gene (CRISPR/Cas) system. Common bovine targets for genetic modification are milk protein genes such as β-lactoglobulin, β-casein, myostatin, horned/polled, prolactin receptor conferring a slick haircoat for improving heat tolerance, and various genes involved in disease susceptibility or resilience (Wang et al., 2022).

Likewise, a “genetically modified cell” refers to a cell where the genomic DNA of the cell has been manipulated to express one or more exogenous genes and/or to introduce mutation(s) within endogenous genes or intergenic regions which affects expression or functional activity of one or more endogenous genes or gene products.

The Zona Pellucida (ZP) prevents attachment of embryonic cells to culture substrates. Accordingly, in an embodiment, the ZP of the embryo is removed prior to contact with the layered ECM-coated substrate and/or outgrowth media. ZP-free embryos can be provided or obtained using any suitable method. For example, the ZP may be thinned and/or ruptured using enzymatic, chemical, and/or mechanical means, and subsequently separated from the embryo by mechanical manipulation to obtain a ZP-free embryo. Suitable enzymatic or chemical means for thinning and/or rupturing the ZP include for example the use of proteases such as pronase or acidified Tyrode's solution. Suitable mechanical methods for rupturing the ZP include for example the use of a microblade, micropipette, microneedle, or laser. The ruptured ZP may be separated from the embryo for example by agitation such as pipetting, vortexing, or direct manipulation using a micropipette or microneedle.

In one embodiment, the ZP-free embryo is obtained by enzyme-assisted ZP removal. As set out in the Examples, protease treatment of a morula (6-day) bovine embryo, followed by mechanical rupture and separation of the ZP results in a ZP-free embryo suitable for deriving naive embryonic stem cells as described herein.

Conventional embryo biopsy techniques result in rupture of the ZP for example using a microblade. Accordingly, in an embodiment the ZP-free embryo is obtained from a biopsied embryo.

Desirable genetic characteristics in embryos may arise as a result of processes such as meiosis, mutation, and the immigration of genes, which can occur naturally or by genetic modification. Accordingly, in an embodiment the ZP-free embryo is obtained from a genetically modified embryo.

Embryos from which the ZP-free bovine embryo is obtained may be fresh or previously frozen, and optionally may be obtained from biopsied-frozen embryos. In an embodiment the embryo is a genetically tested embryo.

In one embodiment, the bovine embryo is a reconstructed diploid embryo. Reconstructed diploid embryos are described, for example in Smith et al., WO 2020/168422, the contents of which is incorporated by reference herein in its entirety.

Diploid embryos with predetermined genomes can be generated in vitro by reconstructing biparental embryos using screened and selected androgenetic and parthenogenetic embryonic haploid cells (Smith et al., WO 2020/168422). Genomes for the reconstructed diploid embryos can be produced to contain a unique combination of alleles, haplotypes, or traits meeting stringent genetic criteria for a large complement of genetic or genomic characteristics such as production traits (e.g. milk, fat, protein, fat %, protein %, milk protein variant composition. g. A2A2 milk), meat quality traits, growth traits, health traits (e.g. somatic cell score, mastitis resistance, immune response, livability, disease resistance), reproductive traits (e.g. pregnancy rate, conception rate), calving traits (e.g. calving ease, calving to first insemination, stillbirths), conformation traits (e.g. polled traits, udder and teat traits, feet and leg traits, body traits, dimension traits), efficiency traits (e.g. feed efficiency traits, workability, longevity, productive life), novel traits (e.g. robotic milking traits, heat tolerance, activity traits and behavior traits), and composite index traits (e.g. LPI (Life Production Index), TPI (Total Production Index), and the absence of various deleterious alleles and haplotypes (e.g. dwarfism, mulefoot, hypotrichosis, brachyspina, citrullinemia, bovine leukocyte adhesion deficiency).

Most cells in culture (except stromal derived cells) require a supporting layer to attach and proliferate in vitro such as in a tissue culture dish. This supporting layer can be made from stromal cells (directly attached on the dish), commonly known as a feeder cell system. For the culture of embryonic stem cells, a mouse embryonic fibroblast (MEF) feeder system is frequently used. However, one disadvantage of using a feeder system is the potential of cross-species contamination when cells, such as embryonic stem cells, from a species different than the mouse is cultured on MEF. Another option is to use a protein matrix as a supporting layer, also known as feeder-free system.

Boggliotti et al. (2018) describe the use of a feeder system for culturing bovine primed embryonic stem cells wherein to compensate for the low attachment rate embryos were pressed to the bottom of the culture dish using a needle. However, use of such exogenous mechanical forces may damage the cells. In contrast, the embodiments described herein achieve attachment and outgrowth formation of less differentiated naïve embryonic stem cells without mechanically pressing the cells onto the culture dish. In one embodiment, the methods and products described herein provide a feeder-free system for the derivation of naïve embryonic stem cells.

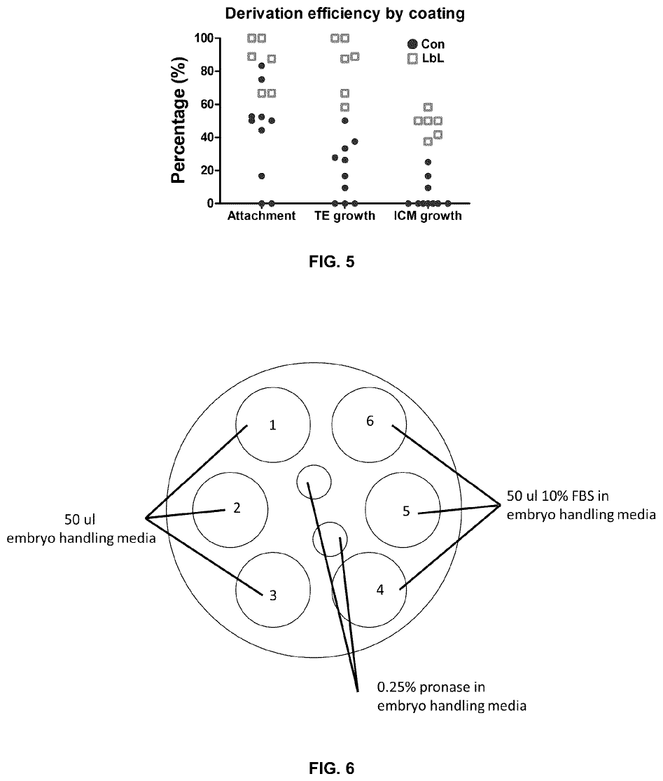

For example, as shown in FIG. 5 an ECM-coated substrate generated using a Layer-by-Layer (LbL) protocol with a positively charged biocompatible polymer binding layer exhibited higher and more stable attachment rates as well as TE (trophectodermal cell) and ICM (inner cell mass) growth compared to a control substrate using ECM extracted from Engelbreth-Holm-Swarm murine sarcoma cells, such as Matrigel (from Corning) or Geltrex (from Invitrogen).

Accordingly, in one embodiment, there is provided a substrate comprising a positively charged surface; a layer of a positively charged biocompatible polymer in contact with the negatively charged surface of the substrate; and a layer of a negatively charged ECM in contact with the layer of the positively charged biocompatible polymer.

Also provided is a method of preparing an ECM-coated substrate suitable for embryo attachment and outgrowth as described herein. In one embodiment the method comprises:
a) providing a substrate comprising a negatively charged surface,
b) contacting the negatively charged surface with a first solution comprising a bio-compatible polymer, wherein the biocompatible polymer is positively charged;
c) incubating the substrate in contact with the first solution such that a layer of the positively charged biocompatible polymer is deposited on the negatively charged surface of the substrate;
d) removing the first solution and optionally washing the substrate;
e) contacting the substrate with a second solution comprising an extracellular matrix (ECM), wherein the ECM is negatively charged; and
f) incubating the substrate in contact with the second solution such that a layer of the negatively charged ECM is deposited on the layer of the positively charged biocompatible polymer.

As used herein, the term “substrate” generally means a physical surface onto which layer(s) of materials are deposited or adhered. The substrate may be rigid or flexible and may be made of any suitable material, for example a plastic such as polystyrene. The substrate may be treated to render it hydrophilic and/or impart a charge such as a negative charge to the surface. Optionally the substrate is plasma-treated. In an embodiment the substrate is plasma-treated polystyrene (also known as tissue culture plastic).

As used herein, the term “biocompatible polymer” generally means a polymer that is compatible with living tissues or cells, for example a polymer which is non-toxic and does not elicit undesirable effects on for example the survival, growth, proliferation and/or other biological activities of cells. Biocompatible polymers may be inert with respect to such activities, and/or may support desired activities. The biocompatible polymer may be a naturally occurring polymer, may be prepared from a naturally occurring polymer, or may be a synthetic polymer with the desired properties. Suitable biocompatible polymers have properties so as to result in deposition and/or adherence of the polymer onto the surface of the substrate under conditions used for coating the substrate with the polymer. Deposition or adherence of the polymer may for example occur through electrostatic interactions, hydrogen bonding, or any other suitable type(s) of interaction(s). Accordingly, suitable properties of the biocompatible polymer may include for example a charge, such as a positive charge, at the pH of the solution used for coating. The interaction between polymer and substrate should be maintained under conditions (e.g. pH) used for subsequent washing and ECM coating steps, as well as conditions used for cell culture (e.g. physiological pH). Suitable polymers include gelatin type A (such as that derived from acid-cured tissue). Accordingly, in an embodiment, the biocompatible polymer is gelatin type A, optionally porcine gelatin type A.

As understood in the art, the terms “incubate” or “incubating” means to maintain for example a substance, material, composition, etc. at a particular temperature, or within a temperature range, for a period of time.

As used herein, the term “physiological pH” means a pH of about 7.1 to about 7.6, optionally about 7.15 to about 7.45, about 7.2 to about 7.4, about 7.25 to about 7.35, or about 7.3.

The term “extracellular matrix” or “ECM” as used herein generally means a biocompatible matrix comprising one or more macromolecule components, such as for example proteins, glycosaminoglycans (GAGs), and/or proteoglycans, which provides attachment and support for the growth and proliferation of cells, such as for example cells grown ex vivo or in vitro. Common ECM components may include, without limitation, one or more of laminin, collagen (e.g. collagen I-XIV), fibronectin, vitronectin, entactin/nidogen, heparan sulfate proteoglycans, and/or one or more functional variants thereof. As known in the art, ECM commonly includes basement membrane extracts such as those isolated from Engelbreth-Holm-Swarm (EHS) mouse sarcoma cells, and hereinafter referred to as “EHS-ECM”, (for example sold under trade names Matrigel™ (Corning) and Geltrex™ (Thermo Fisher)). In an embodiment, the EHS-ECM may comprise for example laminin, collagen IV, entactin/nidogen, and heparan sulfate proteoglycans. Alternatively, other sources of ECM with different compositions and/or purified components (e.g. FN), and/or synthetic ECM-like substrates (e.g. comprising RGD peptides) could also be used in the coating methods if desired. As will be understood by the skilled person, the choice of suitable ECM component(s) will depend on a number of factors including without limitation, cell type, stage of differentiation, and other experimental parameters. For example, EHS-ECM is demonstrated herein to be suitable for culture (e.g. attachment and outgrowth) of bovine embryos. Accordingly, in an embodiment, the ECM is EHS-ECM, optionally Matrigel or Geltrex.

As shown herein, the attachment and outgrowth formation from bovine embryos is influenced by the composition of the outgrowth medium in which the embryos are cultured. The outgrowth medium may comprise for example a base medium, and one or more small molecules, growth factors, and/or nutrients. Suitable base media can be readily determined by the skilled person and includes without limitation DMEM/F12, advanced DMEM/F12 and Neurobasal medium. Suitable supplements can be readily determined by the skilled person and include without limitation MEM non-essential amino acids, L-glutamine, Glutamax, ascorbic acid, insulin, BSA (fraction V), beta-mercaptoethanol and penicillin/streptomycin. In one aspect of the disclosure there are provided outgrowth media components useful for deriving and maintaining naïve embryonic stem cells including the attachment and outgrowth of the ICM. In one embodiment, the outgrowth media comprises a base medium and/or supplements as well as one or more outgrowth medium components. In an embodiment, the outgrowth medium comprises one or more of an N2B27 component comprising B27 supplement and N2 supplement, optionally comprising about 1% B27 supplement and about 0.5% N2 supplement; a Wnt activator component, optionally CHIR99021, BIO, CHIR-98014, LY2090314, or IM-12; a Wnt inhibitor component, optionally XAV939, IWR-1, or IWP-2; a MEK/ERK inhibitor component, optionally PD0325901, Ravoxertinib, GSK1120212, MEK162, PD184352, Trametinib, LY3214996, or Ulixertinib; a ROCK inhibitor component, optionally Y27632, Thiazovivin, or Blebbistatin; a LIF component, optionally human LIF; a PKC inhibitor, optionally Gö6983, Gö6976, LY317615, LY333531, PKC412, GSK690693, Sotrastaurin, Staurosporine, or Bisindolylmaleimide; and an insulin component, optionally insulin. Optionally, the outgrowth medium may comprise one or more of N2B27, CHIR99021 (a Wnt activator), XAV939 (a Wnt inhibitor), PD0325901 (a MEK/ERK inhibitor), Ravoxertinib (ERK specific inhibitor) Gö6983 (PKC inhibitor), Y27362 (ROCK inhibitor), and/or LIF. In an embodiment, the outgrowth media further comprises an Activin A component, optionally human Activin A.

### II. Products and Compositions of Matter

In one aspect of the disclosure there are provided products and compositions of matter useful for the derivation and culture of naïve bovine embryonic stem cells.

For example, in one embodiment there is provided an ECM-coated substrate comprising a substrate comprising a negatively charged substrate surface adjacent to a positively charged biocompatible polymer layer and a negatively charged ECM layer adjacent to the positively charged biocompatible polymer layer. In one embodiment, the biocompatible polymer is type A gelatin. In one embodiment, the ECM comprises EHS-ECM. In one embodiment, the substrate is a plastic substrate suitable for cell culture such as polystyrene.

Also provided are formulations of media suitable for promoting bovine embryo attachment, outgrowth formation and/or for deriving naïve bovine embryonic stem cells as described herein.

In one embodiment, the outgrowth medium comprises a base medium and one or more components identified herein. For example, in one embodiment, the outgrowth medium comprises one or more of an N2B27 component, a Wnt activator component, a Wnt inhibitor component, a MEK/ERK inhibitor component, a ROCK inhibitor component, a LIF component, a PKC inhibitor, and an insulin component. In one embodiment, the outgrowth medium, comprises an N2B27 component, a Wnt activator component, a Wnt inhibitor component, a MEK/ERK inhibitor component, a ROCK inhibitor component, a LIF component, a PKC inhibitor, and an insulin component.

In one embodiment, the N2B27 component comprises B27 supplement and N2 supplement, optionally about 1% B27 supplement and about 0.5% N2 supplement.

In one embodiment, the Wnt activator component comprises CHIR99021, BIO, CHIR-98014, LY2090314, or IM-12. Optionally, the Wnt activator component comprises CHIR99021, optionally at a concentration of about 0.1 uM to about 5 uM, optionally about 1 uM, to about 3 uM, optionally about 1 uM, about 2 uM, or about 3 uM.

In one embodiment, the Wnt inhibitor component comprises XAV939, IWR-1, or IWP-2. Optionally, the Wnt inhibitor component comprises XAV939, optionally at a concentration of about 0.2 uM to about 10 uM, optionally about 1 uM to about 5 uM, optionally about 2 uM. Optionally, the Wnt inhibitor component comprises IWR-1, optionally at a concentration of about 0.25 uM to about 10 uM, optionally about 1 uM to about 5 uM, optionally about 2.5 uM.

In one embodiment, the MEK/ERK inhibitor component comprises PD0325901, Ravoxertinib, GSK1120212, MEK162, PD184352, Trametinib, LY3214996, or Ulixertinib. Optionally, the MEK/ERK inhibitor component comprises PD0325901 or Ravoxertinib. Optionally, the MEK/ERK inhibitor component comprises PD0325901 at a concentration of about 0.05 uM to about 5 uM, optionally about 0.1 uM to about 2 uM, optionally about 1 uM. Optionally the MEK/ERK inhibitor component comprises Ravoxertinib at a concentration of about 0.25 uM to about 10 uM, optionally about 1 uM to about 5 uM, optionally about 2.5 uM.

In one embodiment, the ROCK inhibitor component comprises Y27632 Thiazovivin, or Blebbistatin. Optionally, the ROCK inhibitor component comprises Y27632, optionally at a concentration of about 0.5 uM to about 20 uM, optionally about 5 uM to about 10 uM, optionally about 5 uM or about 10 uM.

In one embodiment, the LIF component comprises human LIF, optionally at a concentration of about 1 ng/ml to about 1000 ng/ml, optionally about 5 ng/ml to about 100 ng/ml, optionally about 5 ng/ml, about 10 ng/ml, about 20 ng/ml, or about 100 ng/ml.

In one embodiment, the Activin A comprises human Activin A, optionally at a concentration of about 1 ng/ml to about 50 ng/ml, optionally about 5 ng/ml to about 50 ng/ml, optionally about 10 ng/ml or about 20 ng/ml.

In one embodiment, the PKC inhibitor comprises Gö6983, Gö6976, LY317615, LY333531, PKC412, GSK690693, Sotrastaurin, Staurosporine, or Bisindolylmaleimide. Optionally, the PKC inhibitor comprises Gö6983, optionally at a concentration of about 0.2 uM to about 25 uM, optionally about 2 uM to about 2.5 uM, optionally about 2 uM or about 2.5 uM.

In one embodiment, the insulin component comprises insulin peptide, optionally at a concentration of about 2 μg/ml to about 200 μg/ml, optionally about 20 μg/ml.

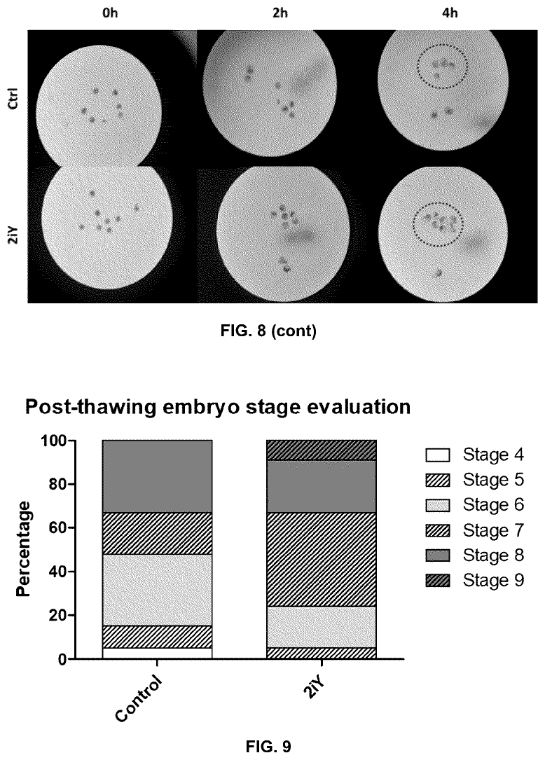

Also provided are formulations suitable for the post-thawing recovery of embryos. As shown in FIGS. 8 and 9, embryos treated with a recovery media formulated as described herein exhibited a faster recovery and a higher derivation efficiency as observed by the faster re-expansion of the embryos relative to controls. Embryos treated with recovery media also appeared to be larger and had a clearer ICM relative to controls.

In one embodiment, the recovery media comprises a glycogen synthase kinase 3 (GSK-3) inhibitor, a MEK/ERK kinase inhibitor, and a Rho-associated, coiled-coil containing protein kinase (ROCK) inhibitor. Suitable GSK-3 inhibitors include for example CHIR99021. Suitable MEK/ERK inhibitors include for example PD0325901. Suitable ROCK inhibitors include for example Y27632. In an embodiment, the recovery medium comprises CHIR99021, optionally 0.1 uM to about 5 uM, optionally about 1 uM, to about 3 uM, optionally about 1 uM, about 2 uM, or about 3 uM; PD0325901, optionally about 0.05 uM to about 5 uM, optionally about 0.1 uM to about 2 uM, or about 1 uM; and Y27632, optionally about 0.5 uM to about 20 uM, optionally about 5 uM to about 10 uM, or about 10 uM.

Also provided are kits comprising one or more of an ECM-coated substrate, an outgrowth media and/or a recovery media as described herein. In one embodiment, the ECM-coated substrate, outgrowth media and/or recovery media are packaged in separate containers.

An aspect includes a naïve bovine stem cell derived using the methods described herein. In an embodiment, the naïve bovine stem cell is a genetically modified stem cell.

### III. Uses

Also provided is the use of the products, compositions or kits described herein for supporting the attachment of embryos in culture, outgrowth of the embryonic cells, and/or derivation of naïve embryonic stem cells.

For example, in one embodiment there is provided use of an ECM-coated substrate as described herein for culturing embryos and/or cells derived from embryos. In one embodiment, the ECM-coated substrate is useful for promoting the attachment and/or outgrowth of embryos as well as the derivation of naïve embryonic stem cells, optionally naïve bovine embryonic stem cells.

In another embodiment, there is provided use of an outgrowth media as described herein for culturing embryos and/or cells derived from embryos. In one embodiment, the outgrowth medium is useful for promoting for the attachment and/or outgrowth of embryos, as well as the derivation of naïve embryonic stem cells, optionally naïve bovine embryonic stem cells. In one embodiment, the outgrowth media is for use in combination with an ECM-coated substrate as described herein.

In another embodiment, there is provided use of a recovery media or adaptation media as described herein for treating embryos, optionally bovine embryos and optionally reconstructed diploid bovine embryos. In one embodiment, the recovery media is useful for treating fresh embryos, or embryos that have previously been biopsied and/or frozen. Recovery media is also useful for processes in which embryos can benefit from recovery media such as, for example, ZP removal, thawing, biopsy, gene editing, or cell derivation.

A further aspect includes use of a naïve bovine stem cell derived using the methods described herein in a breeding scheme or genetic improvement program, or for multiplying preimplantation embryos having desirable genetic characteristics, deriving primordial germ cells and/or gametes for in vitro breeding programs, and/or developing and delivering veterinary medical biologicals and therapeutics.

The following non-limiting examples are illustrative of the present application:

### EXAMPLES

### Example 1. Optimization of Coating Materials

Three different extracellular matrix coating materials were tested as coating material in this example: Gelatin type A (Sigma, G1890), Geltrex™ (Thermo Fisher, A1413301) and Matrigel™ (Corning, 354277). Unless specifically indicated, EHS-ECM (Extra Cellular Matrix) is used in the Examples to refer to Geltrex and/or Matrigel.

The attachment and outgrowth rate were determined for each type of coating material, and at various concentrations of material.

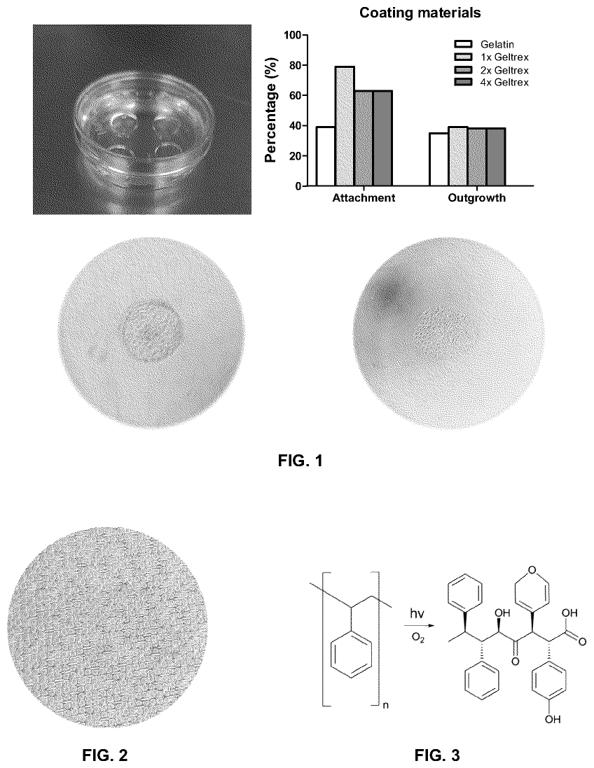

The protocol for coating was as follows:
1. 4 drops of 50 μL of coating solution: 0.1% gelatin or different concentrations of EHS-ECM, provided as a 100× concentrated solution, were applied onto Nunc surface 35 mm² cell culture dish (see FIG. 1).
2. Coated substrates were incubated in a humidified 5% CO₂ incubator at 38.5° C. for 1 hour.
3. After incubation, excess coating solution was removed, 50 uL of cell culture media was applied to the coated area, covered with 4 mL of mineral oil, and 50 ul of additional media was added into each drop.
4. Media was allowed to equilibrate in a humidified 5% O₂, 5% CO₂ incubator at 38.5° C. for 2 hours.
5. After ZP removal and adaptation of embryos, 1 embryo was placed into 1 micro culture drop to start outgrowth derivation.
6. First attachment rate was determined at 48 hours of outgrowth culture and first media change was done right after first attachment determination.
7. Attachment rate and outgrowth rate were evaluated every day and media were changed right after daily evaluation.

### Results

As shown in FIG. 1, higher attachment rates were observed with EHS-ECM coating (Geltrex) compared to gelatin coating, but no difference was observed for the outgrowth formation rate.

No difference in outgrowth efficiency was observed when different concentrations of EHS-ECM were used. Overall attachment rates were poor, resulting in low outgrowth efficiency.

### Example 2. Grooved Surface Coating with Mesh

It was hypothesized that that the smooth surface made by the thin layer of coating solution was not providing a proper “landscape” for the embryo to attach. To test whether the attachment rate could be improved, a grooved surface was created by coating mesh membranes with gelatin or EHS-ECM coating materials.

The protocol for dish fabrication was as follows:
1. Cell strainer membrane (30 um pore size) was cut into 1 cm² size.
2. Cut membrane was placed on a 35 mm² dish.
3. 50 ul of coating materials (0.5% gelatin or 1×EHS-ECM) was applied to the cut membrane. 1×EHS-ECM gave the best attachment rate in Example 1 and was therefore selected for this experiment.
4. Coating material was allowed to polymerize in the humidified incubator at 38.5° C. for 1 hour and then allowed to dry on the bench (under the laminar flow hood) at room temperature for 30 min.
5. Coated surface was sterilized under UV for 20 min.
6. The surface fabricated dish was placed on ice and the membrane carefully removed.

### Results

As shown in FIG. 2, a grooved dish surface can easily be made with this protocol. However, the grooves collapsed rapidly following the addition of culture media.

### Example 3. ECM-Coated Substrates Generated Using a Layer-by-Layer (LbL) Protocol

Even though with moderate attachment rates were obtained with EHS-ECM when fresh embryos were used, outgrowths were difficult to derive, especially from frozen-thawed embryos. Using the coating protocol described in Example 1, some protein debris was observed at the bottom of the culture drop. It was hypothesized that protein matrix had detached from the surface of culture dish, and further hypothesized that the protein matrix was not attached strongly enough to the culture dish. To address this problem, the coating protocol was amended to employ a layer-by-layer approach.

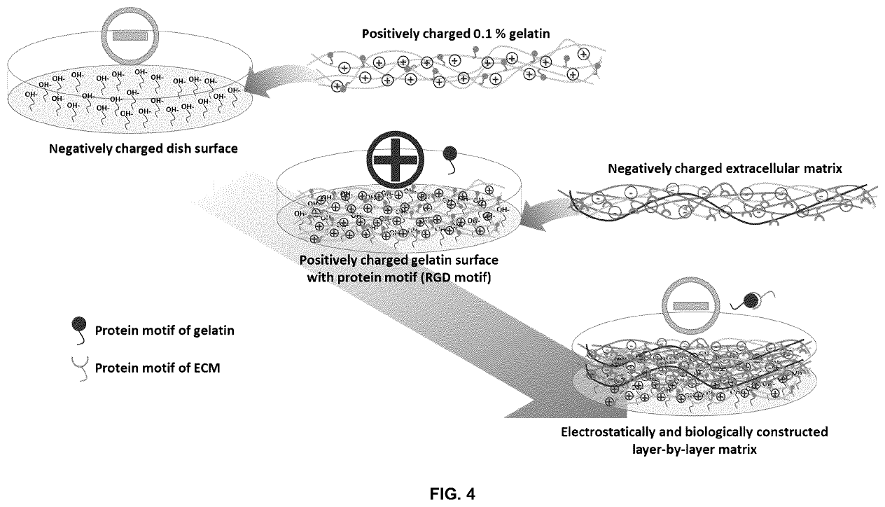

The Nunc™ delta (plasma treated) polystyrene dishes used in Examples 1 and 2 have a negatively charged surface (see e.g. FIGS. 3 and 4). Common ECM components, for example basement membrane extracts, are also negatively charged under conditions used for coating and/or at physiological pH. For example, Geltrex (A14133, Thermo fisher) is negatively charged at pH 7.2, which is the pH of the diluent buffer used for coating (isoelectric point of EHS-ECM: 4-5). The molecules of a protein matrix can be adhered with plasma treated polystyrene surfaces by hydrogen binding with the —OH residue of the surface (Lerman et al., 2018).

However, since both components have similar electrostatic charge, interaction between both sides is relatively weak. Therefore, a “Layer-by-Layer” approach was investigated, wherein a positively charged material is positioned as an “electrostatic glue” between both negatively charged components (see e.g. FIG. 4). Gelatin type A (commonly extracted from porcine) has an isoelectric point of 7-9, while Gelatin type B (commonly extracted from bovine) has an isoelectric point of 4.8-5.1. Therefore, Gelatin type A is positively charged at pH 7.2, making it a suitable candidate to act as an “electrostatic glue” in a Layer-by-Layer protocol. In addition, gelatin can not only provide electrostatic stability, but also provide plenty of protein motifs similar to EHS-ECM, which may reinforce the biological interaction between the gelatin layer and the EHS-ECM layer.

The protocol for generating LbL ECM coated substrate was as follows:
1. 40 ul of 0.1% Gelatin type A (diluted with DMEM/F12 basal medium) was applied to the surface of a Nunc 35 mm² cell culture dish to make small drops.
2. The dish was incubated in a humidified 5% CO₂ incubator at 38.5° C. for 1 hour.
3. After incubation, the gelatin solution was removed and 50 ul of DPBS with Ca²⁺ and Mg²⁺ was added to wash excess gelatin from drops, followed by removal using aspiration. The washing step was repeated 2 times more.
4. 50 ul of 1×EHS-ECM was applied to the gelatin coated area and incubated in a humidified 5% CO₂ incubator at 38.5° C. for 1 hour.
5. After incubation, EHS-ECM solution was removed, and 50 ul of media was applied; drops were covered with 4 ml of mineral oil, and 50 ul of additional media was added into each drop.
6. The media was allowed to equilibrate in 5% CO₂ incubator for 2 hours.

### Results

As shown in FIG. 5, the attachment of embryos was improved and more stable using a LBL EHS-ECM coated substrate compared to the control condition (EHS-ECM only). The use of LbL EHS-ECM coated substrate also drastically improved the ability to produce outgrowth from embryos. Outgrowths exhibited better ICM growth, which ultimately gives rise to embryonic stem cells, compared to regular EHS-ECM coated substrates. An improvement of TE growth was also observed relative to controls.

### Example 4. Embryo Preparation for Outgrowth Culture

The embryo, also called blastocyst, is surrounded by a thick membrane of glycoprotein which forms the zona pellucida (ZP). During development, the blastocyst will start to expand and ultimately “hatch”, which will allow the blastocyst to get out of that “shell”. It is possible to obtain hatching and hatched blastocysts generally around day-8 of in vitro culture. However, bovine in vitro embryo culture media can efficiently support the development until day-7, after which the embryo needs to be either transferred into a recipient or frozen. From preliminary experiments, the ZP is found to block the attachment of embryo, so it should be removed prior to outgrowth culture. Therefore, investigations were performed to try and improve ZP removal from either fresh day-6/day-7 and/or frozen/thawed day-7 embryos.

### Enzyme-Based Zona Pellucida (ZP) Digestion Protocol for Fresh Embryos

Enzyme-based approach is a frequently used technique for ZP removal. A concentration of Protease (from Streptomyces griseus, Pronase, Sigma P8811) between 0.05 to 0.5% is commonly used for that protocol. However, since protease is not a glycoprotein specific enzyme, it can also damage the embryo once the ZP has been completely digested.

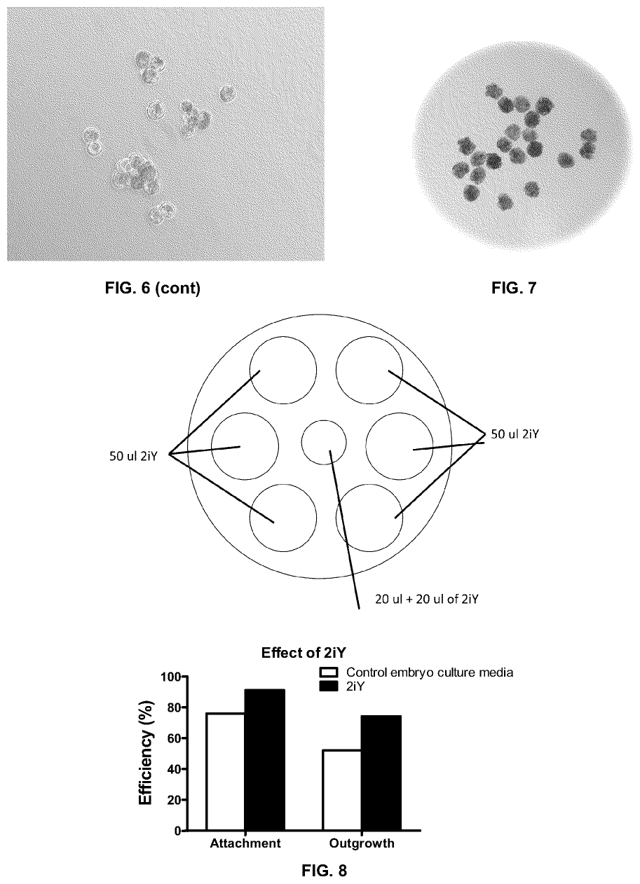

The ZP digestion protocol was as follows:
1. A protease treatment dish was prepared (see FIG. 6).
2. An embryo was transferred from the embryo culture media to drop 1 of embryo handling media (Hepes-buffered media).
3. Embryos were washed by successive transfers from drop 1 to 3.
4. Embryos were transferred into the protease drop (concentrations tested: 0.05%; 0.1%; 0.25% or 0.5%) and incubated until digestion of ZP was observed (about 2 min).
5. Protease-treated embryos were transferred to drop 4, 5, and 6 of inactivation medium made with 10% FBS in embryo handling media.

### Results

As shown in FIG. 6, the extended exposure to protease can cause dissociation of the embryo itself and damage the cells (as illustrated by dark and nontransparent cells).

Since morula stage (e.g. bovine day-6) embryos have very thick ZP (compared to expanded blastocyst stage (e.g. bovine day-7) where the ZP has started to become thinner because of the expansion of the blastocyst), complete digestion was not possible at the lowest concentrations (<0.1%).

With higher concentration (>0.25%), enzyme was not completely inactivated after exposure to FBS, so embryos were negatively affected by high concentrations of protease, which resulted in a complete dissociation of the embryo into single blastomere.

The timing required to digest the ZP is also highly variable between embryos. Therefore, a standardized protocol would have been very difficult to obtain.

### Enzyme-Assisted ZP Removing Protocol for Fresh or Frozen/Thawed

Existing embryo biopsy protocols for day-7 embryos employ the use of a micro-blade to cut through the ZP to perform the embryo biopsy. The opening in the ZP created by the biopsy facilitates release of the embryo from the ZP. However, this approach cannot be applied easily to morula stage (e.g. day-6) embryos due to the much thicker/harder ZP. Therefore, two different protocols were combined to develop an “enzyme-assisted ZP removing protocol” for morula stage embryos. Briefly, embryos are treated with protease sufficient to make the ZP thin and soft without causing dissociation of the embryo or damage to the cells. Then, morula with a thinned ZP, can now easily be used with the regular biopsy technique using a micro-blade.

ZP digestion protocol was as follows:
1. A protease dish was prepared with 0.25% protease (e.g. as shown in FIG. 6). Protease drop was prepared a maximum of 2 hours before use.
2. One or more embryos were transferred from embryo culture medium to drop 1 made with embryo handling media.
3. Embryos were washed by transferring from drop 1 to 3.
4. Embryos were transferred into 0.25% protease drop and incubate for 45 seconds.
5. Transfer treated embryos to drop 4, 5, and 6 of inactivation medium made with 10% FBS in embryo handling media.
6. Transfer ZP thinned embryos into biopsy dish.

### Results

Enzyme assisted ZP removing protocol can be used to produce ZP-free morulas as shown in FIG. 7.

### Example 5. Compositions to Improve Post-Thaw Quality of Biopsied Frozen Embryos

In breeding applications, biopsied-frozen embryos are typically genetically identified (e.g. screened for biomarkers), allowing practitioners to select those have desired genetic characteristics. Even though it is possible to biopsy and freeze day-7 embryos, those embryos are exposed to more stress compared to fresh embryos. Biopsied-frozen embryos have opened ZP (because of the biopsy procedure), therefore ZP can be easily removed by gentle pipetting. However, biopsied-frozen-thawed embryo quality is generally inferior to fresh embryos. Herein, a post-thawing recovery medium (called “2iY” media or “recovery media” herein) is described, which includes three inhibitors: CHIR99021, PD0325901 and Y27632. CHIR99021 and PD0325901 are well characterized inhibitors, also known as “2i” in the stem cell field. Those two inhibitors modulate two important pathways involved in transcription factor activity of naïve embryonic stem cells (CHIR99021: Wnt pathway activator though inhibition of GSK3, PD0325901: MEK pathway inhibition). Y27632, which is a ROCK inhibitor, is an actin filament stabilizer. The ability of these three inhibitors to protect the cells from the post-thawing stress and to promote a faster cell recovery by modulating key stemness pathways was tested. The experiments shown herein demonstrate that this protocol significantly improves the quality of the embryos post-thaw, which allows for more efficient derivation of out-growths.

### Protocol

1. 3 uM CHIR99021 (2520691, PeproTech), 1 uM PD0325901 (3911091, PeproTech), and 10 uM Y27632 (1293823, PeproTech) was added to embryo handling media (2iY medium).
2. A thawing dish was prepared using 2iY media (see FIG. 8) or control embryo culture media.
3. One or more embryos were thawed according to protocol.
4. Embryos were put into the 2iY medium or control embryo handling media.
5. ZP was removed by gentle pipetting right after thawing.
6. Embryos were incubated in a humidified incubator with 6.8% CO₂, 5% O₂ at 38.5° C. for 4 hours.
7. Embryos were transferred to outgrowth medium when fully recovered (re-expanded). Recovery was confirmed by the presence of the blastocyst cavity appearance.

### Results

As shown in FIG. 8, the 2iY treated group exhibited higher attachment and outgrowth forming rate than the control group when plated for 2 days and 4-5 days, respectively, on LbL-ECM coated dishes. 2iY treated embryos demonstrate faster recovery than control embryo culture media and higher derivation efficiency as observed by the faster re-expansion of the embryos. 2iY treated embryos are larger and show a clear ICM relative to the control group (dashed circles in FIG. 8) after 4 hours of recovery.

2iY treated group exhibits more advanced stage and improved quality of embryos 4 hours after thawing from cryopreservation (see FIG. 9).

### Example 6. Adaptation of Embryos to New Culture Environment

Cells in culture, especially embryonic stem cells, are very sensitive to media components and nutrients levels. However, there are two factors that are frequently overlooked in cell culture especially when complete/commercial media are used, namely media osmolarity and pH. Transferring the embryos directly from embryo handling media to DMEM (embryonic stem cells basal medium) was observed to be stressful on cells (data not presented) and osmolarity and pH measurements confirmed the wide differences between both media (see Table 1). Therefore, it was hypothesized that an adaptation medium may facilitate gradual adaptation to of cells to a new microenvironment.

### Protocol

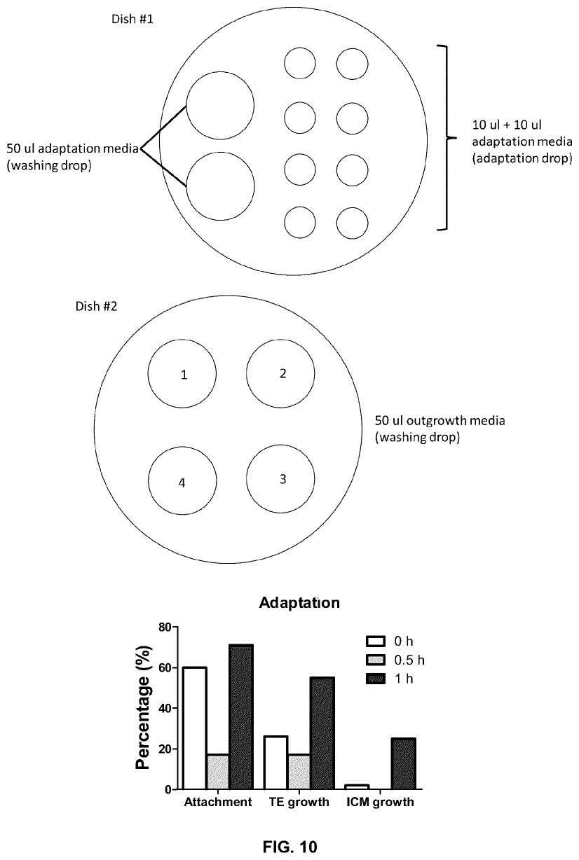

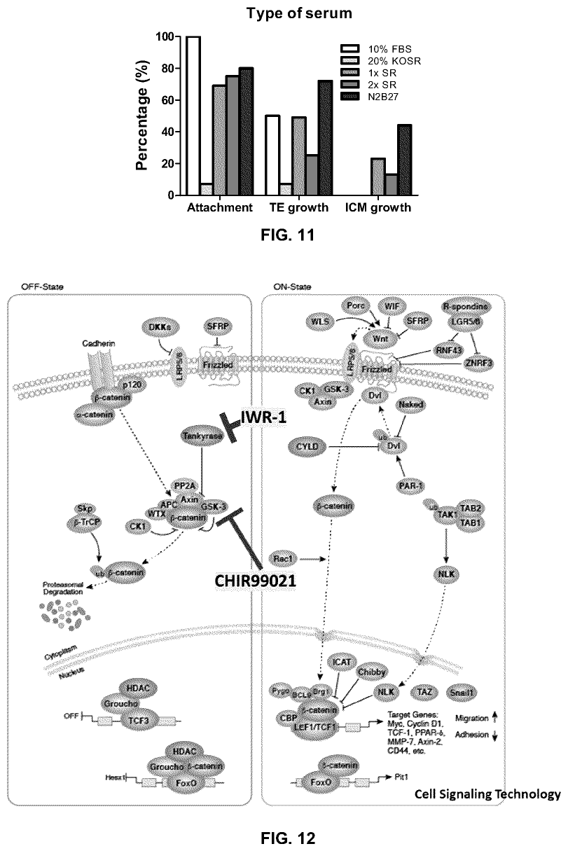

1. Thawing media and outgrowth media was mixed at a 1:1 ratio.
2. Adaptation dishes were prepared as shown in FIG. 10.
3. After post-thaw recovery, embryos were washed twice in adaptation media (1:1 ratio of thawing media (e.g. embryo handling media and outgrowth media (e.g. DMEM))) before being put in the adaptation drop in the Dish #1 (FIG. 10).
4. After 1 hour of adaptation, embryos were then washed four times by transferring to washing drops (outgrowth media) in the Dish #2.
5. Embryos were then moved to the outgrowth media (DMEM) for plating.
6. Adapted embryos were then plated into LbL coated micro drop.

### Results

TABLE 1
Measurements of pH and osmolarity (Osm) for various
media. Measurements were obtained in duplicate.

| Media | pH | Osm |
|---|---|---|
| Embryo handling media | 7.17, 7.15 | 269, 268 |
| Adaptation | 7.21, 7.22 | 309, 310 |
| Outgrowth media | 7.27, 7.28 | 353, 353 |

The efficiency of derivation (attachment, TE growth, and ICM growth measured 4-6 days after the embryo plating) as a function of adaptation treatment is shown in FIG. 10. After 1 hour of adaptation, the efficiency of derivation is improved compared to 0.5 hours of adaptation or controls with no adaptation. More than 1 hour of adaptation (2 and 4 hour incubations) did not shown any difference relative to a 1 hour adaptation.

### Example 7. Testing of Culture Media for Ability to Support Outgrowth Formation

Cell culture media contain various components to support general maintenance of cells such as metabolism, survival and proliferation. Additional growth factors or inhibitors may also be added to promote differentiation, self-renewal or simply boost cell growth. To derive naïve embryonic stem cells and maintain them in an undifferentiated state, requires a combination of growth factors and inhibitors in the culture media. Improper combinations of additives or different concentration of those molecules can induce irreversible differentiation of stem cells.

### Type of Serum/Serum Replacement

As shown in FIG. 11, five different serum sources/concentrations conditions were tested. FBS (10091, Gibco™), Knock-out Serum Replacement (10828-10, Gibco™), Serum Replacement (S0638, Sigma), B27 supplement (17504-044, Gibco™), and N2 supplement (17502-048, Gibco™) were used for this test. Traditional mouse 2iL media (DMEM/F12+1% MEM non-essential amino acid+2 mM Glutamax+100 UM beta-mercaptoethanol+100 IU penicillin/streptomycin+growth factors/inhibitors including 3 UM CHIR99021, 1 μM PD0325901 and 100 ng/ml LIF) was used as a basal medium for the serum tests of 10% FBS and 20% KOSR, and 1×-, 2×-SR groups. t2iLGöY media (1:1 mixture of DMEM/F12 and neurobasal medium+1% MEM non-essential amino acid+2 mM Glutamax+50 μg/ml BSA, fraction V+100 μM beta-mercaptoethanol+100 IU penicillin/streptomycin+growth factors/inhibitors including 1 μM CHIR99021, 1 μM PD0325901, 10 μM Y27632, 2.5 μM Gö6983 and 10 ng/ml LIF) was used for the testing of N2B27 group. The reason for that different combination is because the 2iL medium has a much simpler formulation (compared to t2iLGoY medium), which makes it more suitable for basic serum sources such as FBS; KOSR or SR.

Knock-Out Serum Replacement (KOSR) which is widely used serum source in other species has shown very low efficiency with bovine embryo outgrowth.

Serum replacement (SR), which contains bovine origin components, has shown much better efficiency compared to KOSR.

N2B27 media, which includes 1% of B27 supplement and 0.5% N2 supplement, exhibited the best result for bovine embryo outgrowth derivation (TE and ICM growth) compared to the other components tested.

### Additional Base Media and Other Components/Additives Tested

Traditional mouse 2iL media: Media composition: DMEM/F12+1× serum replacement+100 ng/ml leukemia inhibitory factor (LIF)+3 μM CHIR99021+1 μM PD0325901.

TABLE 2
Derivation rates using mouse 2iL media

| | | | Growth | | | | |
|---|---|---|---|---|---|---|---|
| Media | Basal media | Origin | factors/inhibitors | n= | Attach % | TE % | ICM % |
| 2iL | DMEM/F12 | Day 6 | hLIF: 100 ng/ml | 32 | 88% | 50% | 19% |
| | | embryo | CHIR99021: 3 μM | | | | |
| | | | PD0325901: 1 μM | | | | |

A positive attachment rate was obtained, but outgrowth forming rate was low with only 19% of ICM expansion and 50% TE expansion.

Naïve Human Stem cell Media—NHSM (Gafni et al., 2013): Media composition: DMEM/F12: Neurobasal media (1:1 mixture)+N2B27 serum+8 ng/ml FGF+1 ng/ml TGF-b+20 ng/ml LIF+3 uM CHIR99021+1 uM PD0325901+10 uM SP600125+10 uM+SB203580

TABLE 3
Derivation rates using NHSM media

| | | | Serum | Growth | | | | |
|---|---|---|---|---|---|---|---|---|
| Media | Basal media | Origin | Type | factors/inhibitors | n= | Attach % | TE % | ICM % |
| NHSM | 1:1 mixture of | Day 5 | N2B27 | hFGF: 8 ng/ml | 30 | 63% | 27% | 0% |
| | DMEM/F12 and | embryo | | hTGF-b: 1 ng/ml | | | | |
| | Neurobasal | | | hLIF: 20 ng/ml | | | | |
| | medium | | | CHIR99021: 3 μM | | | | |
| | | | | PD0325901: 1 μM | | | | |
| | | | | SP600125: 10 μM | | | | |
| | | | | SB203580: 10 μM | | | | |

This combination exhibited very low efficiency with 0% ICM expansion.

### Forskolin Media

Forskolin is an adenylyl cyclase stimulator and increases CAMP level in the cells which is secondary messenger involved in many signaling pathways. The combination of forskolin and 2iL media revealed good efficiency in the derivation of human naïve stem cells (Hanna et al., 2010) and the reprogramming of bovine naïve-like pluripotent stem cell (Kawaguchi et al., 2015).

Formulation of 2iLFk: Traditional Mouse 2iL Media+10 uM Forskolin

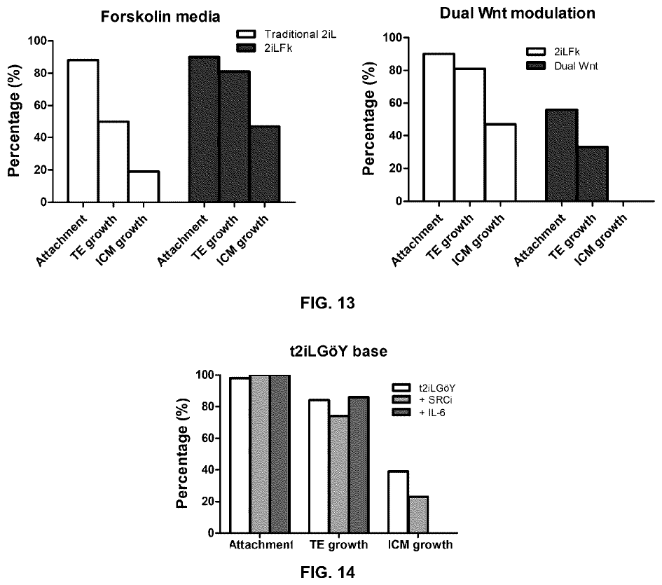

This combination showed an improved derivation efficiency compared to traditional 2iL media and NHSM media (FIG. 13). However, ICM derived naïve stem cell colonies did not show any further expansion in 2iLFk media after passaging.

### Dual Kinase Modulation of Wnt

Wnt is known to be a key player for naïve stem cell signaling pathways. The combination of Wnt activator CHIR99021 and Wnt inhibitor such as IWR-1 or XAV939 (both inhibitor of the same protein complex, but each are targeting a different unit) cause cytoplasmic accumulation of beta-catenin and can promote self-renewal of mouse pluripotent stem cells through stabilization of E-cadherin which is key component of adherent junctions (Kim et al. 2013). The ability of the dual modulation of Wnt to overcome the poor expansion observed with 2iLFk media was tested.

Formulation of Dual Wnt media: 1.5 uM CHIR99021+2.5 uM IWR-1+1 uM PD0325901+100 ng/ml LIF+10 uM Forskolin.

As shown in FIG. 13, this combination showed a negative effect with bovine embryonic stem cells.

### Bovine IL-6 (Super Family of LIF) and SRCi:

Bovine IL-6 (superfamily of LIF) and SRC inhibitor were tested as a replacement for hLIF and PD0325901, respectively, used in t2iLGoY media, which has been used for human naïve stem cell derivation (titrated 2i/LIF/Gö6983/Y27632, Guo et al., 2016).

Human LIF was initially used in the formulation of t2iLGoY. However, the sequence of human and bovine LIF are different. Therefore, bovine IL-6, which is a member of the LIF superfamily, was tested as a replacement of human LIF.

SRC inhibitor, which is an RTK inhibitor, is involved in most of the signaling pathways induced by growth factors (Theunissen et al., 2014). Knowing that the endpoint of SRC inhibition would ultimately target the ERK/MEK pathway, a SRC inhibitor was tested as an alternative and novel strategy to the MEK pathway inhibition (though the MEKi PD0325901).

Formulation of t2iLGöY media: DMEM/F12: Neurobasal media (1:1 mixture)+N2B27 serum+1 uM CHIR99021+1 uM PD0325901+10 ng/ml human LIF+2.5 uM Gö6983+10 uM Y27632.

Formulation of SRCi media: 12iLGoY media where PD0325901 has been replaced by 2 uM of CGP77675 (SRC inhibitor) and human LIF was replaced by 10 ng/ml bovine IL-6.

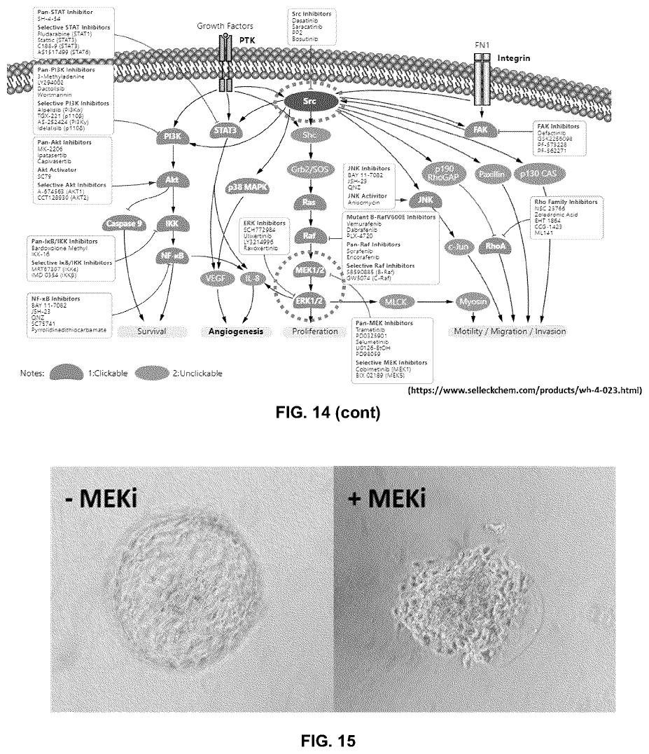

As shown in FIG. 14, substitution of either bovine IL-6 for human LIF or SRC inhibitor CGP77675 for MEK inhibitor PD0325901 showed a negative effect on derivation efficiency compared to t2iLGöY media, either through low attachment or low outgrowth formation rates.

### MEK Inhibitor (MEKi)

Zhao et al., (2021) published results suggesting that MEK inhibitor must be removed from bovine ES cell media, which is in contrast with previously published results regarding human cells. Investigations were performed to test that approach by removing the MEK inhibitor from the media.

TABLE 4
Derivation rates with MEK modulation.

| | | | Serum | Growth | | | |
|---|---|---|---|---|---|---|---|
| Media | Basal media | Origin | Type | factors/inhibitors | Attach % | TE % | ICM % |
| +MEK | 1:1 mixture of | Day 6 | N2B27 | CHIR99021: 1 μM | 98% | 83% | 34% |
| inhibitor | DMEM/F12 and | embryo | | PD0325901: 1 μM | | | |
| | Neurobasal | | | Gö6983: 2.5 μM | | | |
| | medium | | | Y27632: 10 μM | | | |
| | | | | hLIF: 20 ng/ml | | | |
| −MEK | 1:1 mixture of | Day 6 | N2B27 | CHIR99021: 1 μM | 67% | 50% | 0% |
| inhibitor | DMEM/F12 and | embryo | | Gö6983: 2.5 μM | | | |
| | Neurobasal | | | XAV939: 2 μM | | | |
| | medium | | | hLIF: 20 ng/ml | | | |

When MEK inhibitor, PD0325901 is withdrawn from media, derivation efficiency is decreased, as shown by the embryo cavity formation which is a clear sign of ICM cell differentiation (see FIG. 15).

### PKC Inhibitor, Gö6983

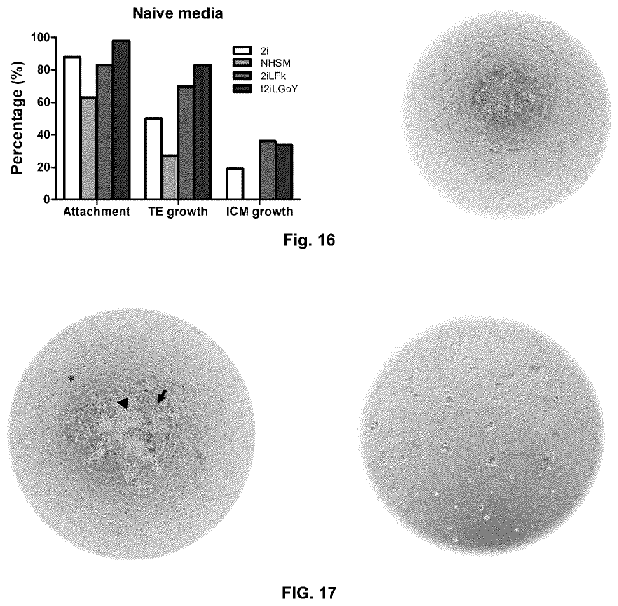

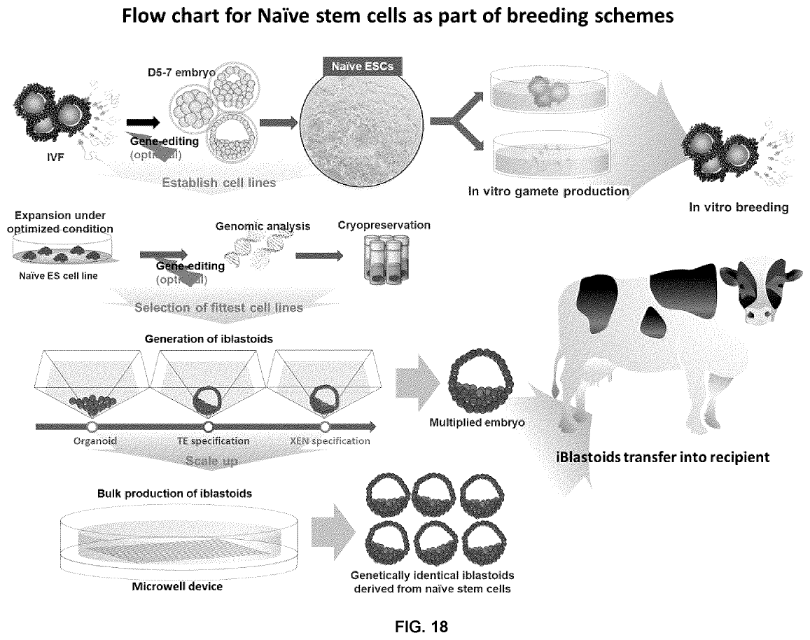

With the recent refinements of the culture media composition, the best culture conditions tested to date, namely the 2iLFk and the t2iLGoY media were tested side-by-side with the 2i and the NHSM media. Remarkably, as shown in FIG. 16, the addition of the PKC inhibitor Gö6983 (Guo et al., 2016) in the t2iLGoY improved the derivation efficiency.

t2iLGoY medium showed similar ICM growth rate but much higher rate in attachment and outgrowth formation, especially TE growth.

Formulation of t2iLGoY: DMEM/F12: Neurobasal media (1:1 mixture)+N2B27 serum+1 uM CHIR99021+1 uM PD0325901+10 ng/ml LIF+2.5 uM Gö6983+10 uM Y27632.

### Insulin

ICM cell morphology was not maintained after first passage using various media compositions described above. ICM cells quickly differentiated into extraembryonic endoderm cells (hypoblast) during outgrowth culture and after passaging.

It was hypothesized that adjusting the concentration of insulin in the media might overcome that type of differentiation (Anderson et al., 2017).

With the addition of 20 μg/ml of insulin to the medium, hypoblast formation is not observed during outgrowth culture and ICM cells can be obtained after passaging, as shown in FIG. 17.

### ERK Pathway Inhibition

Khan et al. (2021) used high-throughput chemical screening to identify the best culture conditions for human naïve stem cells, and inhibition of ERK pathway was identified as a key factor to maintain stable human naïve stem cells culture (Khan et al., 2021).

Inhibition of Wnt pathway has also been emphasized as a key factor in the human naïve stem cells field (Bredenkamp et al., 2019). The approaches from Khan et al. (2021) and Bredenkamp et al. (2019) were combined to investigate and develop the PXGRY/LA medium.

Formulation of PXGRY/LA: DMEM/F12: Neurobasal media (1:1 mixture)+N2B27 serum+1 uM PD0325901+2 uM XAV939+2 uM Gö6983+2.5 uM Ravoxertinib+10 uM Y27632+10 ng/ml LIF+20 ng/ml ActivinA.

As shown in Table 5, the activation of Wnt pathway (with the t2iLGoY medium) or its inhibition (though the use of the PXGRY/LA medium) did not show any significant difference in the efficiency to derive naïve outgrowths using a feeder-free system. However, naïve stem cells are generally derived and maintained on feeder cells, and naïve stem cell media is technically designed for such culture systems. According to Cosin-Roger et al., 2019 and Talbot et al., 2012, feeder cells secrete several important growth factors and more importantly Wnt ligands. In PXGRY/LA medium with feeder-free system, the Wnt pathway is not only inhibited but also completely depleted from Wnt ligands, which are normally secreted from feeder cells. The effects of Wnt inhibition may therefore not be the same using feeder-free conditions compared with cells maintained on feeder cells, especially after several passages of culture. t2iLGöY may therefore be more suitable for bovine naïve cell derivation using feeder-free conditions.

TABLE 5
Derivation rates with Wnt modulation.

| | | | Serum | Growth | | | |
|---|---|---|---|---|---|---|---|
| Media | Basal media | Origin | Type | factors/inhibitors | Attach % | TE % | ICM % |
| t2iLGöY | 1:1 mixture of | Day 6 | N2B27 | CHIR99021: μM | 98% | 83% | 34% |
| | DMEM/F12 and | embryo | | PD0325901: 1 μM | | | |
| | Neurobasal | | | Gö6983: 2.5 μM | | | |
| | medium | | | Y27632: 10 μM | | | |
| | | | | hLIF: 20 ng/ml | | | |
| PXGRY/LA | 1:1 mixture of | Day 6 | N2B27 | PD0325901: 1 μM | 100% | 90% | 33% |
| | DMEM/F12 and | embryo | | XAV939: 2 uM | | | |
| | Neurobasal | | | Gö6983: 2 μM | | | |
| | medium | | | Ravoxertinib: 2.5 uM | | | |
| | | | | Y27632: 10 μM | | | |
| | | | | hLIF: 20 ng/ml | | | |
| | | | | Activin A: 20 ng/ml | | | |

### Example 8: Production of iBlastoid Structures

Stable bovine naïve stem cells generated using the high efficiency protocols described in the preceding Examples are used to reconstruct iBlastoid structures using an approach similar to what was recently published by Liu et al. (2021), Yu et al. (2021), and Yanagida et al. (2021).

### Protocol

1. Dissociate naïve stem cells colony into single cells using enzymatic dissociation methods.
2. Count the number of cells and adjust cell density to 500 cells/ml in iBlastoid media 1. iBlastoid Media 1 will allow the differentiation of naïve stem cell into trophoblast stem cells, to form the outer layer (TE) of the blastocyst.
3. Plate 100 cells (200 ul) into each well of non-adherent round-bottom 96-well plate with multichannel pipette.
4. After 48 hours, remove iblastoid media 1 from the culture plate and add 200 ul of iBlastoid media 2.
5. At 48 hours, transfer well-established iblastoid into iblastoid manipulating media (Hepes buffered DMEM/F12+Neurobasal media) and evaluate iblastoid quality based on the regular embryo grading system.
6. Freeze iblastoid by Boviteq embryo freezing protocol.

Formulation of iBlastoid media 1 is Advanced-DMEM/F12+0.5% Serum replacement+1% MEM NEAA+1% Glutamax™+0.1 mM beta-mercaptoethanol+Gentamycin+2 μM CHIR99021+5 μM Y27632+0.5 mM Valproic acid+1 μM A83-01+50 ng/ml EGF.

Formulation of iBlastoid media 2 is 1:1 mixture of DMEM/F12 and 10 neurobasal medium+1% MEM NEAA+1% Glutamax™+0.1 mM beta-mercaptoethanol+100 IU/ml Pen/Strep+100 ng/ml Activin A+3 μM CHIR99021+10 ng/ml LIF.

### REFERENCES

Anderson K G V, Hamilton W B, Roske F V., Azad A, Knudsen T E, Canham M A, Forrester L M and Brickman J M (2017) Insulin fine-tunes self-renewal pathways governing naive pluripotency and extra-embryonic endoderm. Nature Cell Biology 19 1164-1177.
Bishop T F and Van Eenennam A L. (2020) Genome editing approaches to augment livestock breeding programs. J Exp Biol. 223 (Suppl_1): jeb207159 . . . doi: 10.1242/jeb.207159.
Bogliotti Y S, Wu J, Vilarino M, Okamura D, Soto D A, Zhong C, Sakurai M, Sampaio R V, Suzuki K, Izpisua Belmonte J C et al. (2018) Efficient derivation of stable primed pluripotent embryonic stem cells from bovine blastocysts. Proceedings of the National Academy of Sciences of the United States of America 115 2090-2095.
Bredenkamp N, Stirparo G G, Nichols J, Smith A and Guo G (2019) The Cell-Surface Marker Sushi Containing Domain 2 Facilitates Establishment of Human Naive Pluripotent Stem Cells. Stem Cell Reports 12.
Cosin-Roger J, Ortiz-Masià M D and Barrachina M D (2019) Macrophages as an emerging source of wnt ligands: Relevance in mucosal integrity. Frontiers in Immunology 10 2297.
De Los Angeles A (2019) Frontiers of Pluripotency. Methods in Molecular Biology 2005 3-27.
Gafni O, Weinberger L, Mansour A A, Manor Y S, Chomsky E, Ben-Yosef D, Kalma Y, Viukov S, Maza I, Zviran A et al. (2013) Derivation of novel human ground state naive pluripotent stem cells. Nature 504.
Guo G, Von Meyenn F, Santos F, Chen Y, Reik W, Bertone P, Smith A and Nichols J (2016) Naive Pluripotent Stem Cells Derived Directly from Isolated Cells of the Human Inner Cell Mass. Stem Cell Reports 6.
Hanna J, Cheng A W, Saha K, Kim J, Lengner C J, Soldner F, Cassady J P, Muffat J, Carey B W and Jaenisch R (2010) Human embryonic stem cells with biological and epigenetic characteristics similar to those of mouse ESCs. Proceedings of the National Academy of Sciences of the United States of America 107 9222-9227.
Hou Z, An L, Han J, Yuan Y, Chen D and Tian J (2018) Revolutionize livestock breeding in the future: an animal embryo-stem cell breeding system in a dish. Journal of Animal Science and Biotechnology 2018 9:1 9 1-11.
Kawaguchi T, Tsukiyama T, Kimura K, Matsuyama S, Minami N, Yamada M and Imai H (2015) Generation of Naïve Bovine Induced Pluripotent Stem Cells Using PiggyBac Transposition of Doxycycline-Inducible Transcription Factors. PLOS ONE 10 e0135403.
Khan S A, Park K mi, Fischer L A, Dong C, Lungjangwa T, Jimenez M, Casalena D, Chew B, Dietmann S, Auld D S et al. (2021) Probing the signaling requirements for naive human pluripotency by high-throughput chemical screening. Cell Reports 35.
Kim H, Wu J, Ye S, Tai C I, Zhou X, Yan H, Li P, Pera M and Ying Q L (2013) Modulation of β-catenin function maintains mouse epiblast stem cell and human embryonic stem cell self-renewal. Nature Communications 4.
Lerman M J, Lembong J, Muramoto S, Gillen G and Fisher J P (2018) The Evolution of Polystyrene as a Cell Culture Material. Tissue Engineering—Part B: Reviews 24.
Li R, Zhong C, Yu Y, Liu H, Sakurai M, Yu L, Min Z, Shi L, Wei Y, Takahashi Y et al. (2019) Generation of Blastocyst-like Structures from Mouse Embryonic and Adult Cell Cultures. Cell 179 687-702.e18.
Liu X, Tan J P, Schröder J, Aberkane A, Ouyang J F, Mohenska M, Lim S M, Sun YBY, Chen J, Sun G et al. (2021) Modelling human blastocysts by reprogramming fibroblasts into iBlastoids. Nature 591 627-632.
Park F. (2007) Lentiviral vectors: are they the future of animal transgenesis? Physiol Genomics. 31:159-173.
Smith, L C, Labrecque, R, Paredes LMA, Therrien J, and Vigneault, C. 2020. Use of haploid embryonic cells to generate offspring with predetermined genomes. WO 2020/168422.
Solter D and Knowles B B (1975) Immunosurgery of mouse blastocyst. Proc Natl Acad Sci USA 72 (12): 099-102
Soto D A, Navarro M, Zheng C, Halstead M M, Zhou C, Guiltinan C, Wu J and Ross P J (2021). Simplification of culture conditions and feeder-free expansion of bovine embryonic stem cells. Scientific Reports 2021 11:1 11 1-15.
Talbot N C, Sparks W O, Powell A M, Kahl S and Caperna T J (2012) Quantitative and semiquantitative immunoassay of growth factors and cytokines in the conditioned medium of STO and CF-1 mouse feeder cells. In Vitro Cellular & Developmental Biology. Animal 48 1-11.
Theunissen T W, Powell B E, Wang H, Mitalipova M, Faddah D A, Reddy J, Fan Z P, Maetzel D, Ganz K, Shi L et al. (2014) Systematic identification of culture conditions for induction and maintenance of naive human pluripotency. Cell Stem Cell 15.
Wang S, Qu Z, Huang Q, Zhang J, Lin S, Yang Y, Meng F, Li J, and Zhang K. (2022) Application of Gene Editing Technology in Resistance Breeding of Livestock. Life (Basel). 12:1070. doi: 10.3390/life12071070.
Yanagida A, Spindlow D, Nichols J, Dattani A, Smith A and Guo G (2021) Naive stem cell blastocyst model captures human embryo lineage segregation. Cell Stem Cell 28.
Yu L, Wei Y, Duan J, Schmitz D A, Sakurai M, Wang L, Wang K, Zhao S, Hon G C and Wu J (2021) Blastocyst-like structures generated from human pluripotent stem cells. Nature 591 620-626.
Zhao L, Gao X, Zheng Y, Wang Z, Zhao G, Ren J, Zhang J, Wu J, Wu B, Chen Y et al. (2021) Establishment of bovine expanded potential stem cells. Proceedings of the National Academy of Sciences of the United States of America 118.

1. A method of deriving naïve bovine embryonic stem cells, the method comprising:
a) providing a Zona Pellucida (ZP)-free bovine embryo comprising naïve bovine embryonic stem cells;
b) contacting the ZP-free bovine embryo with an extracellular matrix (ECM)-coated substrate, wherein the ECM-coated substrate comprises a substrate comprising a negatively charged substrate surface adjacent to a positively charged biocompatible polymer layer, and a negatively charged ECM layer adjacent to the positively charged biocompatible polymer layer; and
c) culturing the ZP-free bovine embryo in the presence of outgrowth medium to induce attachment of the ZP-free bovine embryo to the ECM-coated substrate and outgrowth of an inner cell mass (ICM) comprising derived naïve bovine embryonic stem cells.

2. The method of claim 1, wherein the positively charged biocompatible polymer layer comprises type A gelatin.

3. The method of claim 1, wherein the negatively charged ECM layer comprises EHS-ECM.

4. The method of claim 1, wherein the substrate comprises polystyrene.

5. The method of claim 1, wherein the outgrowth medium comprises one or more of:
an N2B27 component;
a Wnt activator component;
a Wnt inhibitor component;
a MEK/ERK inhibitor component;
a ROCK inhibitor component;
a LIF component;
a Activin A component
a PKC inhibitor; and
an insulin component.

6. The method of claim 5, wherein the outgrowth medium comprises:
the N2B27 component;
the Wnt activator component;
the Wnt inhibitor component;
the MEK/ERK inhibitor component;
the ROCK inhibitor component;
the LIF component;
the PKC inhibitor; and
the insulin component.

7. The method of claim 6, wherein the outgrowth medium further comprises an Activin A component, optionally human Activin A.

8. The method of claim 5, wherein
the N2B27 component comprises B27 supplement and N2 supplement, optionally about 1% B27 supplement and about 0.5% N2 supplement;
the Wnt activator component comprises CHIR99021, BIO, CHIR-98014, LY2090314, and/or IM-12;
the Wnt inhibitor component comprises XAV939, IWR-1, and/or IWP-2;
the MEK/ERK inhibitor component comprises PD0325901, Ravoxertinib, GSK1120212, MEK162, PD184352, Trametinib, LY3214996, and/or Ulixertinib;
the ROCK inhibitor component comprises Y27632, Thiazovivin, and/or Blebbistatin;
the LIF component comprises human LIF;
the PKC inhibitor comprises Gö6983, Gö6976, LY317615, LY333531, PKC412, GSK690693, Sotrastaurin, Staurosporine, and/or Bisindolylmaleimide; and/or
the insulin component comprises insulin.

9. The method of claim 1, wherein the ZP-free bovine embryo is a 3- to 8-day embryo, optionally a 6- or 7-day embryo, or the ZP-free embryo is a morula (stage 4); a blastocyst (stage 5); an expanding blastocyst (stage 6); an expanded blastocyst (stage 7); a hatching blastocyst (stage 8) or a hatched blastocyst (stage 9).

10. The method of claim 1, wherein the ZP-free bovine embryo is obtained by enzyme-assisted ZP removal.

11. The method of claim 10, wherein the method further comprises obtaining the ZP-free bovine embryo by enzyme-assisted ZP removal comprising the steps of:
a) providing an embryo;
b) contacting the embryo with a protease solution;
c) incubating the embryo in the protease solution to partially digest the ZP and obtain a ZP-thinned embryo;
d) contacting the ZP-thinned embryo with a protease inactivation medium to inactivate the protease;
e) rupturing the ZP; and
f) manipulating the embryo to separate the ZP from the embryo.

12. The method of claim 11, wherein the concentration of protease in step c) is 0.1%-0.5%, optionally about 0.25%.

13. The method of claim 11, wherein the embryo and protease solution are incubated for between about 30-60 seconds in step c), optionally for about 45 seconds.

14.-29. (canceled)

30. A method of preparing an extracellular matrix (ECM)-coated substrate, the method comprising:
a) providing a substrate comprising a negatively charged surface;
b) contacting the negatively charged surface with a first solution comprising a biocompatible polymer, wherein the biocompatible polymer is positively charged;
c) incubating the substrate in contact with the first solution such that a layer of the positively charged biocompatible polymer is deposited on the negatively charged surface of the substrate;
d) removing the first solution and optionally washing the substrate;
e) contacting the substrate with a second solution comprising an extracellular matrix (ECM), wherein the ECM is negatively charged; and
f) incubating the substrate in contact with the second solution such that a layer of the negatively charged ECM is deposited on the layer of the positively charged biocompatible polymer.

31. The method of claim 30, wherein the biocompatible polymer is type A gelatin.

32. The method of claim 30, wherein the ECM comprises EHS-ECM.

33. The method of claim 30, wherein the substrate is polystyrene.

34.-41. (canceled)

42. A media composition comprising:
an N2B27 component;
a Wnt activator component;
a Wnt inhibitor component;
a MEK/ERK inhibitor component;
a ROCK inhibitor component;
a LIF component;
a PKC inhibitor; and
an insulin component.

43. The media composition of claim 42, wherein
the N2B27 component comprises B27 supplement and N2 supplement, optionally about 1% B27 supplement and about 0.5% N2 supplement;
the Wnt activator component comprises CHIR99021, BIO, CHIR-98014, LY2090314, or IM-12;
the Wnt inhibitor component comprises XAV939, IWR-1, or IWP-2;
the MEK/ERK inhibitor component comprises PD0325901, Ravoxertinib, GSK1120212, MEK162, PD184352, Trametinib, LY3214996, or Ulixertinib;
the ROCK inhibitor component comprises Y27632, Thiazovivin, or Blebbistatin;
the LIF component comprises human LIF;
the PKC inhibitor comprises Gö6983, Gö6976, LY317615, LY333531, PKC412, GSK690693, Sotrastaurin, Staurosporine, or Bisindolylmaleimide; and/or
the insulin component comprises insulin.

44. The media composition of claim 42, further comprising an Activin A component, optionally human Activin A.

45.-57. (canceled)
